(19) United States
(12) Patent Application Publication
(10) Pub. No.: US 2011/0070214 A1
(43) Pub. Date: Mar. 24, 2011

(54) BACITRACIN METAL COMPLEXES USED AS BLEACH CATALYSTS

(75) Inventors: Thomas Weber, Dormagen (DE); Ralf Weidenhaupt, Erkelenz (DE); Karl-Heinz Maurer, Erkrath (DE)

(73) Assignee: HENKEL AG & CO. KGaA, Dusseldorf (DE)

(21) Appl. No.: 12/955,400

(22) Filed: Nov. 29, 2010

Related U.S. Application Data
(63) Continuation of application No. PCT/EP2009/055996, filed on May 18, 2009.

(30) Foreign Application Priority Data
Jun. 9, 2008 (DE) 102008027375

(51) Int. Cl. A01N 63/02; C11D 3/395; A01P 1/00; D06L 3/00; C12N 9/00; D21C 9/10

(52) U.S. Cl. 424/94.1; 510/276; 8/107; 435/183; 435/264; 162/72

(57) ABSTRACT

Bacitracin, bacitracin derivatives and other oligopeptides, alone or in the form of oligopeptide-metal ion complexes, are shown to be useful as bleach catalysts incorporated in washing and cleaning agents for the cleaning of soiled hard surfaces and textile fabrics. These catalysts may be used in conjunction with hydrogen peroxide or any alternate bleaching agent, or used simply in the presence of atmospheric oxygen to bleach stains. New and novel oligopeptides and their metal ion complexes are disclosed herein, along with methods for the cleaning of hard surfaces and textile fabrics with washing and cleaning agents incorporating these bleach catalysts.

### CROSS-REFERENCE TO RELATED APPLICATIONS

This application is a continuation of PCT Application Serial No. PCT/EP2009/055996, filed on May 18, 2009, which claims priority to 10 2008 027 375 (DE), filed on Jun. 9, 2008.

### FIELD OF THE INVENTION

The present invention relates to bacitracin-metal complexes and related compounds and to their use as bleach catalysts, particularly in washing and cleaning agents.

### BACKGROUND OF THE INVENTION

If bleaching with hydrogen peroxide is to be effective, the peroxide must be converted into a species with greater bleaching action. One possibility for producing activated peroxy compounds involves using peracid precursors, commonly referred to as “bleach activators.” One such example is TAED, which is converted into the active species by perhydrolysis.

A further possibility for producing activated species involves enzyme catalyzed perhydrolysis of carboxylic acid esters or nitrile compounds using perhydrolases.

It is also known to use bleach catalysts for producing activated species, where a bleach catalyst refers to a substance capable of improving the bleaching performance of hydrogen peroxide on a bleachable substance without participating stoichiometrically in the reaction.

The use of bleach catalysts rather than other bleach activation methods has the advantage that sub-stoichiometric amounts are sufficient, resulting in a savings in volume and weight when formulating the bleach-containing product. A reduction in weight, especially in washing and cleaning applications, is associated with a reduction in discharge to the environment. Besides the ecological advantage, a savings in transport and packaging costs may also result.

It should be borne in mind that when bleach activators such as nitriles or TAED are used in an aqueous environment, premature hydrolysis may result. This problem may largely be avoided when bleach catalysts are used. Furthermore, the presence of acid arising from the peracid in non-catalytic bleach activation results in a shift of pH that may have an unfavorable impact on bleaching performance. Moreover, the bleaching performance of most bleach activators is often inadequate at low temperatures.

Metal complexes of organic ligands, such as salenes, saldimines, tris[salicylideneaminoethyl]amines, monocyclic polyazaalkanes, cross-bridged polycyclic polyazaalkanes, terpyridines, and tetraamido ligands, have been described as bleach catalysts. A disadvantage of these metal complexes, which is particularly seen at low temperature, is that they do not exhibit adequate bleaching performance. Alternatively, when these materials give adequate bleaching performance, unwanted color fading and damage to the textile fibers can occur.

Furthermore, complex organic ligands are conventionally produced from non-renewable raw materials such as petroleum and coal. These production methods are associated with the disadvantage of consuming irreplaceable resources that will no longer be available for future generations. Therefore, there is a general requirement to produce complex ligands predominately or even entirely from renewable raw materials. For example, citric acid is currently biotechnologically produced on a large scale from fungal cultures. As a chelant, citric acid is capable of complexing metal. However, such citric acid-metal complexes are not of any particular use as bleach catalysts, for example in washing and cleaning applications.

For the reasons discussed above, bleach catalysts are presently of greater interest than other bleach activation methods, and in principle there is a need for novel bleach catalysts preferably produced from renewable raw materials. Ligands in a bleach catalyst should ideally comprise natural products or derivatives that may be straightforwardly produced from natural materials. The need exists for novel bleach catalysts that can enable effective cleaning of hard surfaces and textile fabrics, preferably without concomitant damage to the hard surfaces or the textile fabrics or their colors. Such novel bleach catalysts should also exhibit sufficient storage stability to be compatible with washing and cleaning agents.

### SUMMARY OF THE INVENTION

In an exemplary embodiment of the present invention, bacitracin-metal complexes are used as bleach catalysts.

In another exemplary embodiment of the present invention, bacitracin-metal complexes are used as bleaching agents in washing and cleaning agents. The prior use of bacitracin-metal complexes as bleaching agents in washing and cleaning agents, and/or the use thereof for cleaning textiles or dishes, has not been described in the prior art and is therefore novel.

In another exemplary embodiment of the present invention, bacitracin-metal complexes surprisingly enable effective cleaning of textile fabrics without excessive damage of the textile fabric, while being very gentle on the colors of the textile fabrics.

### BRIEF DESCRIPTION OF THE DRAWINGS

FIG. 1 illustrates the structure of Bacitracin-A using the three-letter symbols for the common amino acids, with indication of D- or L-isomers, and where Orn represents the uncommon amino acid ornithine.

### DETAILED DESCRIPTION OF THE INVENTION

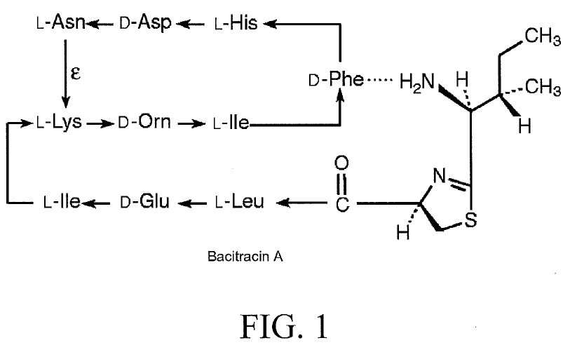

Bacitracin is a pharmaceutical and a polypeptide antibiotic that inhibits cell wall synthesis in some species of bacteria. As shown in FIG. 1, bacitracin-A consists of the following dodecapeptide structure: L-Ile(1)-L-Cys(2)-L-Leu(3)-D-Glu(4)-L-Ile(5)-Lys(6)-D-Orn(7)-L-Ile(8)-D-Phe(9)-L-His(10)-D-Asp(11)-L-Asn(12), wherein the L-Cys(2) and L-Ile(1) residues form a thiazoline ring, and wherein the C-terminal carboxy group of the L-Asn(12) forms an intrachain amide bond with the L-Lys(6) residue such that residues 6-12 comprise a cyclic heptapeptide.

Variants of this major component (bacitracin-A) have also been described which, relative to bacitracin-A, comprise the following substitutions: bacitracin B₁ (Ile8Val), bacitracin B₂ (Ile5Val), bacitracin C₁ (Ile5Val, Ile8Val), bacitracin C₂ (Ile1Val, Ile8Val), bacitracin C₃ (Ile1Val, Ile5Val), bacitracin D_{1b} (Leu3Val, Ile5Val, Ile8Val), and bacitracin E (Ile1Val, Ile5Val, Ile8Val). (Pittenauer et al. (2006) J. Mass Spectrom. 41, 421-447).

Bacitracin is processed as a zinc salt in aqueous tinctures, ointments, pastes or as a powder, and is used in large areas or severely contaminated wounds such as bite, stab, abrasion and tear wounds, and superficial skin infections, inflammation of the outer ear, and burns at risk of infection.

Wishing not to be bound by any theory, it is believed that formation of metal complexes having bleaching action is attributable to the presence of the heterocycles thiazoline and imidazole in bacitracin.

The present invention provides washing and cleaning agents that contain an oligopeptide comprising (a) at least one, optionally completely or partially hydrogenated, thiazole residue, which is preferably at least one thiazoline residue, and (b) at least one, optionally completely or partially hydrogenated, imidazole residue, which is preferably at least one imidazole residue.

Oligopeptides in accordance with the present invention preferably comprise 5 to 30 amino acids, more preferably from 8 to 20, and most preferably from 10-15. Above all, the oligopeptides particularly comprise 10, 11, 12, 13, 14, or 15 amino acids.

Polypeptides according to the present invention preferably comprise at least one intrachain covalent linkage between two amino acids having no alpha-peptide linkages between them. The intrachain linkage preferably consists of an epsilon-peptide linkage of the C-terminal carboxy group of the oligopeptide with an amino group of a lysine side chain. Alternatively, linkage of a carboxyl group of an aspartic acid or glutamic acid side chain with an amino acid group of a lysine side chain may also be useful in the present invention.

The intrachain covalent linkage gives rise to a cyclic peptide structure. In a preferred embodiment of the present invention, the intrachain covalent linkage forms a cyclic hexa-, hepta-, or octapeptide, preferably a cyclic heptapeptide.

According to the present invention, the optionally partially or completely hydrogenated thiazole residue, and in particular a thiazole residue, is preferably formed through covalent linkage of the sulfur of the thiol group of a cysteine side chain with the alpha-carbon atom of an adjacent amino acid attached by an alpha-peptide bond.

The optionally partially or completely hydrogenated imidazole residue is preferably located in an amino acid side chain, this preferably comprising an imidazole residue of histidine.

Polypeptides according to the present invention preferably comprise at least one amino acid having a carboxyl group or carboxamide group in its side chain. In particular, this may be glutamine, glutamic acid, asparagine, or aspartic acid.

In a preferred embodiment of the present invention, the oligopeptide comprises 10 to 20, and preferably 12, 13, 14, or 15 amino acids, wherein the seven C-terminal amino acids form a cyclic heptapeptide by means of an epsilon-peptide bond of the C-terminal amino acid, and wherein two successive amino acids in the N-terminal region of the oligopeptide form a thiazoline residue outside of the cyclic heptapeptide, preferably by covalent linkage of the sulfur of the thiol group of a cysteine side chain with the alpha-carbon of an adjacent amino acid attached by an alpha-peptide bond.

In a particularly preferred embodiment of the present invention, the oligopeptide is bacitracin, and in particular bacitracin-A, bacitracin B₁, bacitracin B₂, bacitracin C₁, bacitracin C₂, bacitracin C₃, bacitracin D_{1b}, or bacitracin E, or a derivative thereof. Herein, a derivative refers to an oligopeptide that, in reference to bacitracin, is substituted in up to five, more preferably in up to four, three, or two amino acid positions, and most preferably in exactly one amino acid position, and/or, in comparison with bacitracin, additionally comprises N-terminally up to five, more preferably up to four, three, or two amino acids, and most preferably exactly one amino acid, and/or refers to an oligopeptide obtainable from bacitracin by chemical modification such as alkylation, acylation, or glycosylation by corresponding treatment with alkylating, acylating, or glycosylating reagents. Here, the variants of bacitracin are preferably those that comprise substitutions by homologous amino acids.

Due to the low level of fiber and color damage desired, the washing and/or cleaning agent is preferably such an agent with particularly good fiber care and/or color care properties. The cyclic peptide according to the present invention and the metal complex thereof may be used in free form and also in the form of any desired salt.

The present invention accordingly provides the use of oligopeptides, preferably bacitracin or derivatives of it, and in particular in the form of metal complexes, in washing and cleaning agents, and in particular in washing and cleaning agents for cleaning hard surfaces and/or textile fabrics.

Also, the present invention accordingly provides the use of oligopeptides, preferably bacitracin or derivatives of it, and in particular in the form of metal complexes, for cleaning and/or removing soils such as tea, coffee, blood, soot, egg, milk, butter, ink, makeup, lipstick, chocolate, chocolate dessert, olive oil, black currant, blueberry, apple juice, red wine, beetroot, curry, and peanut oil, and mixtures thereof.

It is possible also to catalyze redox reactions other than just bleaching reactions using such novel catalysts. The present invention accordingly provides the use of oligopeptides, preferably bacitracin or derivatives of it, and in particular in the form of metal complexes, for the bleaching of wood pulp and/or raw cotton.

Furthermore, the present invention provides a method for cleaning textile fabrics or hard surfaces using a metal complex of an oligopeptide, particularly a bacitracin-metal complex, where due to the particularly good fiber care and/or color care properties of these metal complexes, the method is preferably a fiber care or color care method.

In a particularly preferred embodiment of the present invention, the oligopeptides herein are used directly in the form of a catalytically active metal complex. However, it is also possible to produce the catalytically active metal complex in situ. To this end, the oligopeptide and metal ion, optionally in the form of any desired salt, may be initially introduced physically separate from one another, and/or may be applied sequentially. In this case, the oligopeptide and metal ion may be combined and the bleach catalyst thus formed immediately upon application. For example, physical separation of the oligopeptide and metal ion may be achieved through use of a multichamber system where the oligopeptide and metal ion are held in different chambers. The metal complex of the oligopeptide may be produced in situ by combining the components from the various chambers of the multichamber system. Alternatively, the oligopeptide and the metal ion may be present in different compartments of a pulverulent washing agent, for example in different granules or in various layers of a multilayer granule. Upon dissolution of the granules during application, the oligopeptide and metal ion may then combine to form the catalytically active complex.

The present invention provides a method for cleaning textile fabrics or hard surfaces using a metal complex of an oligopeptide disclosed herein, and in particular using a bacitracin-metal complex where the complex is produced in situ.

In a preferred embodiment of the present invention, the oligopeptides and/or oligopeptide-metal complexes are used together with a source of oxygen, for example a bleaching agent (or a bleach activator) as explained below in greater detail. However, it has been surprisingly found that the metal complexes herein have a relatively strong cleaning and bleaching action even without the addition of a source of oxygen.

The present invention particularly provides a method for cleaning textile fabrics or hard surfaces using a metal complex of an oligopeptide, and in particular a metal complex of bacitracin or a bacitracin derivative where no bleaching agent and/or bleach activator has been added, such that atmospheric oxygen becomes the sole source of oxygen.

According to the present invention, the cyclic peptides usable herein have a complex formation constant relative to manganese (III) of less than 10⁻³M⁻¹, preferably of less than 10⁻⁷M⁻¹, and most preferably of less than 10⁻²⁰M⁻¹.

Naturally occurring oliogopeptides in accordance with the present invention may be produced by microorganisms, in particular bacteria. The oligopeptides are preferably synthesized by “nonribosomal peptide synthetases” (NRPS). Therefore, polypeptides of the present invention are those whose synthesis preferably proceeds naturally by NRPS. In a further preferred embodiment, the oligopeptide of the present invention is a naturally occurring antibiotic.

NRPS consist of a plurality of modules, generally one module per amino acid present in the synthesized oligopeptide. Each module consists of a plurality of domains that have specific tasks with regard to incorporation each respective amino acid. Apart from the starting module from which the C domain may be absent, a module at least comprises the domains C (condensation, selective bond formation), A (identification and activation of the amino acid to be incorporated) and T (temporary fixation of the activated amino acid as a thioester). Further domains that are optionally present include the domains E (racemization of the amino acid for incorporation as a D-amino acid), Z (cyclization of side chains) and TE (thioesterase, cleavage of the finished peptide from the last module).

The polypeptides to be used in accordance with the present invention preferably originate naturally from microorganisms, in particular from bacteria and fungi, in particular those which naturally contain nonribosomal peptide synthetases (see in this connection Donadio et al. (2007) Natural Product Reports 24(5), 1073-1079). The following may be mentioned by way of example Aspergillus, in particular Aspergillus aculeatus, nidulans, niger, flavipes, fumigatus, ochraceus, rugulosus, sclerotiorum, sydowii and versicolor, Alternaria, in particular Alternaria alternata, mali and tenuis, Aureobasidium, in particular Aureobasidium pullulans, Bacillus, in particular Bacillus brevis, licheniformis, megaterium, pumilus and subtilis, Beauveria, in particular Beauveria bassiana, Cochliobolus, in particular Cochliobolus carbonum, Coleophoma, in particular Coleophoma empetri, Cryptosporiopsis, in particular Cryptosporiopsis quercina, Cylindrocladium, in particular Cylindrocladium scoparium, Diaporthe, in particular Diaporthe toxica, Diheterospora, in particular Diheterospora chlamydosporia, Escherichia, in particular Escherichia coli, Fusarium, in particular Fusarium moniliforme, oxysporum and scirpi, Gliocladium, Hapsidispora, in particular Hapsidispora irregularis, Helicoma, in particular Helicoma ambiens, Helminthosporium, in particular Helminthosporium carbonum, Hirsutella, in particular Hirsutella nivea, Isaria, in particular Isaria cretacea, Metarhizium, in particular Metarhizium anisopliae, Mycelia, in particular Mycelia sterilia, Paecilomyces, in particular Paecilomyces fumosoroseus, Petriella, in particular Petriella guttulata, Penicillium, in particular Penicillium chrysogenum, islandicum and Penicillium sp. NK374186, Phoma, in particular Phoma lingam, Pithomyces, in particular Pithomyces chartarum, cynodontis and sacchari, Pseudallescheria, in particular Pseudallescheria boydii, Streptomyces, in particular Streptomyces orientalis and clavuligerus, Tolypocladium, in particular Tolypocladium niveum and terricola, Ustilago, in particular Ustilago maydis, Ustilaginoidea, in particular Ustilaginoidea virens, Verticillium, in particular Verticillium lamellicola and lecanii, Trichothecium, in particular Trichothecium roseum, and Zalerion, in particular Zalerion arboricola.

On the one hand, the naturally occurring polypeptides may be obtained from the natural producers. However, they may alternatively be produced heterologously in another organism by genetically modifying these organisms by incorporating DNA encoding one or more enzymes involved in oligopeptide synthesis. Chemical synthesis of cyclic peptides to be used according to the invention may furthermore be considered as another alternative.

Apart from the above-stated microorganisms that naturally synthesize oligopeptides in accordance with the present invention, and/or that contain NRPS, production organisms which may be considered for heterologous production of oligopeptides according to the invention are furthermore other fungi, yeasts or bacteria, in particular those whose genus contains representatives which naturally produce NRPS. Examples of microorganisms usable according to the invention which may in particular be mentioned are Acinetobacter, Actinomyces, Aerobacter, Alcaligenes, Aspergillus, Alteromonas, Agrobacterium, Aeromonas, Arthrobacter, Aspergillus, Azotobacter, Bacillus, Bordetella, Brevibacillus, Brucella, Burkholderia, Corynebacterium, Erwinia, Escherichia, Fusarium, Hansenula, Klebsiella, Lactobacillus, Mycobacterium, Neisseria, Neurospora, Penicillium, Pichia, Pseudomonas, Ralstonia, Rhizobium, Rhodotorula, Saccharomyces, Saccharopolyspora, Salmonella, Shewanella, Shigella, Staphylococcus, Streptomyces, Ustilago, Vibrio and Yersinia.

Genetic modification of the producing organisms is relatively straightforward to carry out, there being many and varied possible genetic engineering modifications in order to produce an oligopeptide according to the invention with an adapted structure, purity, yield, or process duration. In this way, a ligand produced biotechnologically may be adapted in a particularly versatile manner to requirements, in comparison to one which was not produced biotechnologically. Appropriately created production organisms may furthermore be used to produce further biomolecules which are advantageous for use or purification of the product, such as example coexpression of export apparatuses or suppression of the coproduction of unwanted accompanying substances. Targeted modification of the biosynthesis of nonribosomal peptides such as bacitracin has been demonstrated, example, by module exchange in a nonribosomal peptide synthetase (Stachelhaus et al. (1995) Science 269 (5220), 69-72). Modification of specificity in a domain by targeted mutagenesis and the consequent production of a modified peptide product is described in Eppelmann et al. (Biochemistry (2002) 41(30), 9718-9726). Module deletion is described by Mootz et al. (JACS (2002) 124(37), 10980-10981). Heterologous production of bacitracin in a Bacillus subtilis which does not normally produce bacitracin is described by Eppelmann et al. (J. Biol. Chem. (2001) 276 (37), 34824-34831). In addition to these literature references, reference is in particular also made with regard to methods usable according to the invention to Konz & Marahiel, Chem. Biol. 6 (1999), R39-R48 and the literature cited therein and to Stevens et al., Drug Development Research 66(1) (2005), 9-18.

With regard to naturally occurring oligopeptides such as bacitracin, it should be emphasized that the ability to produce complex ligands from renewable raw materials by biotechnological methods is particularly sustainable, and opens up notably inexpensive process pathways that are not accessible by conventional chemical synthesis.

A further advantage of naturally occurring oligopeptides is that they are readily biodegradable, since they are absorbed by numerous microorganisms and they are, after all, natural substances. This is particularly advantageous if the complexes are intended to get into the environment or they get there unintentionally. Derivatives of naturally occurring oligopeptides should also be correspondingly readily degradable.

Any desired metal atom in any desired oxidation state may in principle be considered as the central atom for the metal complex. The central atom of the metal complex preferably comprises an element which is selected from Ag, Al, Au, B, Bi, Ce, Co, Cr, Cu, Eu, Fe, Ga, Hg, Mn, Mo, Ni, Pb, Pt, Ru, Ti, U, V, W, Zn and Zr in any desired oxidation number, in particular Ag(I), Al(III), Au(III), B(III), Bi(III), Ce(III), Ce(IV), Co(II), Co(III), Cr(III), Cu(I), Cu(II), Eu(II), Eu(III), Fe(II), Fe(III), Ga(III), Hg(II), Mn(II), Mn(III), Mn(IV), Mo(IV), Mo(VI), Ni(II), Pb(II), Pt(II), Ru(II), Ru(III), Ru(IV), U(IV), V(IV), V(V), W(VI) or Zn(II). The central atom of the metal complex particular preferably comprises a transition metal atom which is preferably selected from the group consisting of Mn, Fe, W, Co, Cr, Zn, Bi, Mo, V, Ce and Cu, above all from the group Co, Mn, Ce, Zn, Bi and Fe, in any desired oxidation number. In one particularly preferred embodiment, the central atom is selected from Fe(III), Co(II), Co(III), Mn(II), Mn(III), Mn(IV), Ce(III), Ce(IV), Zn(II) and Bi(III).

Complexes of bacitracin or a derivative thereof with metal ions of Mn, Fe, Co, Ce, Cr, Zn, Cu, Mo, V, W and Bi may in particular be emphasized according to the invention.

The metal complex may assume the form of any desired salt and optionally also comprise further ligands and/or coligands. Ligands and/or coligands which may be considered are in particular any desired salt anions, phosphonates, amines, polymers, polyols together with cobuilders and surfactants, in particular the cobuilders and surfactants listed by way of example further below. Depending on the charge of the metal-oligopeptide complex, counterions which may be considered are cationic counterions such as in particular alkali metal and alkaline earth metal ions or anionic counterions such as in particular halide ions or the anions of organic acids.

The metal complex may generally be produced simply by mixing a metal salt of the corresponding metal with the corresponding oligopeptide or a salt of the corresponding oligopeptide in an aqueous environment. Emergence of a desired oxidation state may be favored by establishing a suitable redox potential.

The present invention also provides metal complexes of oligopeptides according to the invention, in particular of bacitracin or a derivative thereof, wherein the central atom is selected from Ag, Al, Au, B, Bi, Ce, Co, Cr, Cu, Eu, Ga, Hg, Mn, Mo, Ni, Pb, Pt, Ru, Ti, U, V, W, Zn and Zr in any desired oxidation number, in particular from Ag(I), Al(III), Au(III), B(III), Bi(III), Ce(III), Ce(IV), Co(II), Co(III), Cr(III), Cu(I), Cu(II), Eu(II), Eu(III), Fe(II), Fe(III), Ga(III), Hg(II), Mn(II), Mn(III), Mn(IV), Mo(IV), Mo(VI), Ni(II), Pb(II), Pt(II), Ru(II), Ru(III), Ru(IV), U(IV), V(IV), V(V), W(VI) or Zn(II).

The central atom of the metal complex preferably comprises a transition metal atom which is selected from the group consisting of Mn, W, Co, Cr, Zn, Bi, Mo, V, Ce and Cu, above all from the group Co, Mn, Ce, Zn or Bi, in any desired oxidation number. In one particularly preferred embodiment, the central atom is selected from Co(II), Co(III), Mn(II), Mn(III), Mn(IV), Ce(III), Ce(IV), Zn(II) and Bi(III).

The present invention in particular provides the use of the above-stated oligopeptide-metal complexes with the stated central atoms in washing or cleaning agents, in particular for cleaning textile fabrics or for cleaning hard surfaces.

The present invention also provides the use of the above-stated oligopeptide-metal complexes with the stated central atoms as auxiliaries for cleaning textile fabrics and as auxiliaries for cleaning hard surfaces.

The present invention also provides the use of the above-stated oligopeptide-metal complexes with the stated central atoms for bleaching wood pulp and/or raw cotton.

The present invention also provides washing and cleaning agents, preferably surfactant containing, which contain a metal complex in accordance with the present invention where the oligopeptide of the complex is preferably bacitracin or a derivative thereof.

The washing and cleaning agents according to the invention may be any conceivable type of cleaning agent, both as concentrates and as agents for undiluted use, for use on a commercial scale, in washing machines or in manual washing or cleaning. They include for example washing agents for textiles, carpets or natural fibers and, according to the present invention, are designated as washing agents. They also include for example dishwashing agents for dishwashing machines or manual dishwashing washing or cleaning agents for hard surfaces such as metal, glass, porcelain, ceramics, glazed tiles, stone, coated surfaces, plastics, wood or leather; these are designated cleaning agents according to the present invention. More broadly, sterilizing agents and disinfectants should also be considered to be washing and cleaning agents for the purposes of the invention.

Embodiments of the present invention encompass any presentations of the washing or cleaning agents that are established in the prior art and/or are convenient. These include for example solid, pulverulent, liquid, gel-form or pasty agents, optionally comprising two or more phases, compressed or uncompressed, and they furthermore include extrudates, granules, tablets or pouches, packaged both in large containers and in portions.

In a preferred embodiment of the present invention, the washing or cleaning agents may contain the above-described metal complexes of oligopeptides, in particular of bacitracin or a derivative thereof, in a quantity of up to 5 weight percent (wt. %), preferably 0.001 wt. % to 1 wt. %, and more preferably 0.01 wt. % to 0.5 wt. %. Most preferably the washing or cleaning agents may contain the above-described metal complexes of oligopeptides, in particular of bacitracin or a derivative thereof, in a quantity of 0.01 to 0.25 wt. %. Each weight percent recited is relative to the total weight of the washing or cleaning agent.

In addition to the metal complexes in accordance with the present invention, the washing and cleaning agents may also contain other bleach catalysts. In general, these substances may comprise any desired bleach-boosting transition metal salt or any desired transition metal complex. Transition metals that may be considered for this purpose include Mn, Fe, Co, Ru, Mo, Ti, V, and Cu, in their various oxidation states. Exemplary ligands that complex with these metals are described in the literature and include guanidines, aminophenols, amine oxides, salenes, saldimines, lactams, monocyclic and cross-bridged polycyclic polyazaalkanes, terpyridines, dendrimers, tetraamido ligands, bis- and tetrakis(pyridylmethyl)alkylamines, secondary amines, and polyoxometallates.

In a preferred embodiment, a complex of manganese in oxidation state II, III, IV or V, which preferably contains one or more macrocyclic ligands with the donor functions N, NR, PR, O and/or S, is used as an additional bleach catalyst. Ligands which comprise nitrogen donor functionality are preferably used for this purpose. Herein it is particularly preferred to additionally use a bleach catalyst in accordance with the present invention which, as macromolecular ligands, contain 1,4,7-trimethyl-1,4,7-triazacyclononane (Me-TACN), 1,4,7-triazacyclononane (TACN), 1,5,9-trimethyl-1,5,9-triazacyclododecane (Me-TACD), 2-methyl-1,4,7-trimethyl-1,4,7-triazacyclononane (Me/Me-TACN) and/or 2-methyl-1,4,7-triazacyclononane (Me/TACN). Suitable manganese complexes are for example [Mn^{III}₂(μ-O)₁(μ-OAc)₂(TACN)₂](ClO₄)₂, [Mn^{III}Mn^{IV}(μ-O)₂(μ-OAc)₁(TACN)₂]-(BPh₄)₂, [Mn^{IV}₄(μ-O)₆(TACN)₄](ClO₄)₄, [Mn^{III}₂(μ-O)₁(μ-OAc)₂(Me-TACN)₂]-(ClO₄)₂, [Mn^{III}Mn^{IV}(μ-O)₁(μ-OAc)₂(Me-TACN)₂](ClO₄)₃, [Mn^{IV}₂(μ-O)₃(Me-TACN)₂](PF₆)₂ and [Mn^{IV}₂(μ-O)₃(Me/Me-TACN)₂](PF₆)₂ (OAc═OC(O)CH₃).

When used, the additional bleach catalyst is also preferably present in the washing and cleaning agents of the present invention in a quantity of up to 5 wt. %, more preferably from 0.0025 wt. % to 1 wt. %, and most preferably from 0.01 wt. % to 0.25 wt. %, in each case relative to the total weight of the washing or cleaning agent.

Moreover, in a preferred embodiment, the washing and cleaning agents according to the invention contain bleaching agents that preferably constitute and/or release the substrate for bleach catalysts, although, as has already been mentioned above, catalysis may also be carried out without the addition of bleaching agent. According to the invention, a bleaching agent should be understood to mean, on the one hand, hydrogen peroxide itself and, on the other hand, any compound which releases hydrogen peroxide in an aqueous medium. Among those compounds acting as bleaching agents that release H₂O₂ in water, sodium percarbonate, sodium perborate tetrahydrate and sodium perborate monohydrate are of particular significance. Further usable bleaching agents are for example peroxypyrophosphates, citrate perhydrates and H₂O₂-releasing peracid salts or peracids, such as persulfates or persulfuric acid. The urea peroxyhydrate percarbamide, which may be described by the formula H₂N—CO—NH₂H₂O₂ is also usable. In particular when using the agents for cleaning hard surfaces, for example in automatic dishwashing, they may if desired also contain bleaching agents from the group of organic bleaching agents, although the use thereof is in principle also possible in textile washing agents. Typical organic bleaching agents are diacyl peroxides, such as for example dibenzoyl peroxide. Further typical organic bleaching agents are peroxy acids, with examples including alkylperoxy acids and arylperoxy acids. Preferred representatives are (a) peroxybenzoic acid and the ring-substituted derivatives thereof, such as alkylperoxybenzoic acids, as well as peroxy-α-naphthoic acid and magnesium monoperphthalate, (b) aliphatic or substitutedly aliphatic peroxy acids, such as peroxylauric acid, peroxystearic acid, ε-phthalimidoperoxycaproic acid (phthaloiminoperoxyhexanoic acid, PAP), o-carboxybenzamidoperoxycaproic acid, N-nonenylamidoperadipic acid and N-nonenylamidopersuccinates, and (c) aliphatic and araliphatic peroxydicarboxylic acids, such as 1,12-diperoxycarboxylic acid, 1,9-diperoxyazelaic acid, diperoxysebacic acid, diperoxybrassylic acid, diperoxyphthalic acids, 2-decyldiperoxybutane-1,4-diacid, N,N-terephthaloyldi(6-aminopercaproic acid).

Substances that release chlorine or bromine may also be used as bleaching agents. Examples of suitable materials that release chlorine or bromine and may be considered are heterocyclic N-bromamides and N-chloramides, such as trichloroisocyanuric acid, tribromoisocyanuric acid, dibromoisocyanuric acid and/or dichloroisocyanuric acid (DICA) and/or the salts thereof, with cations such as potassium and sodium. Hydantoin compounds, such as 1,3-dichloro-5,5-dimethylhydanthoin are likewise suitable.

Washing and cleaning agents, and in particular automatic dishwashing agents, preferably contain up to 45 wt. %, more preferably from 1 wt. % to 35 wt. %, and most preferably from 3.5 wt. % to 20 wt. % of the bleaching agent. In particular, 5 wt. % to 15 wt. % of bleaching agent may be used, preferably sodium percarbonate.

The active oxygen content of the washing or cleaning agent, and in particular an automatic dishwashing agent, is preferably between 0.4 and 10 wt. %, more preferably between 0.5 and 8 wt. % and most preferably between 0.6 and 5 wt. %. Particularly preferred washing and cleaning agents have an active oxygen content above 0.3 wt. %, preferably above 0.7 wt. %, particularly preferably above 0.8 wt. %, and in particular above 1.0 wt. %.

As an alternative to, or used simultaneously with the bleaching agents, enzymes capable of producing hydrogen peroxide in situ from other substrates may be used to supply the hydrogen peroxide. These comprise oxidoreductases that are capable of transferring electrons from an organic substrate, for instance glucose, to oxygen as the electron acceptor, so as to permit in situ formation of the desired hydrogen peroxide. The oxidoreductase may be used together with the appropriate organic substrate. Since the soils to be treated may, however, already contain the necessary substrate, the oxidoreductases may also be used without addition of the appropriate substrate.

The hydrogen peroxide-producing oxidoreductase preferably comprises an oxidoreductase that produces hydrogen peroxide by using oxygen as the electron acceptor. Enzymes that may in particular be considered are: oxidoreductases of EC classes EC 1.1.3 (CH—OH as electron donor); EC 1.2.3 (aldehyde or oxo group as electron donor); EC 1.4.3 (CH—NH₂ as donor); EC 1.7.3 (nitrogenous group as donor); and, EC 1.8.3 (S-containing group as donor), with the enzymes of EC class EC 1.1.3 being preferred.

Preferred enzymes are selected from the group consisting of malate oxidase (EC 1.1.3.3), glucose oxidase (EC 1.1.3.4), hexose oxidase (EC 1.1.3.5), cholesterol oxidase (EC 1.1.3.6), galactose oxidase (EC 1.1.3.9), pyranose oxidase (EC 1.1.3.10), alcohol oxidase (EC 1.1.3.13), choline oxidase (EC 1.1.3.17, see in particular WO 04/58955), oxidases for long-chain alcohols (EC 1.1.3.20), glycerol-3-phosphate oxidase (EC 1.1.3.21), cellobiose oxidase (EC 1.1.3.25), nucleoside oxidase (EC 1.1.3.39), D-mannitol oxidase (EC 1.1.3.40), xylitol oxidase (EC 1.1.3.41), aldehyde oxidase (EC 1.2.3.1), pyruvate oxidase (EC 1.2.3.3), oxalate oxidase (EC 1.2.3.4), glyoxylate oxidase (EC 1.2.3.5), indole-3-acetaldehyde oxidase (EC 1.2.3.7), pyridoxal oxidase (EC 1.2.3.8), aryl aldehyde oxidase (EC 1.2.3.9), retinal oxidase (EC 1.2.3.11), L-amino acid oxidase (EC 1.4.3.2), amine oxidase (EC 1.4.3.4, EC 1.4.3.6), L-glutamate oxidase (EC 1.4.3.11), L-lysine oxidase (EC 1.4.3.14), L-aspartate oxidase (EC 1.4.3.16), tryptophan alpha, beta-oxidase (EC 1.4.3.17), glycine oxidase EC 1.4.3.19), urea oxidase (EC 1.7.3.3), thiol oxidase (EC 1.8.3.2), glutathione oxidase (EC 1.8.3.3), and sorbitol oxidase, and from enzymes described for example in DE102005053529. Further preferred enzymes include those that are derived from the list stated above, for example by single or multiple mutations in the coding gene, by evolutive methods, by gene shuffling, or by similar methods and, as a consequence, have obtained a new substrate spectrum.

In a preferred embodiment, the hydrogen peroxide-producing oxidoreductase is one that uses a sugar as electron donor. According to the invention, the hydrogen peroxide-producing and sugar-oxidizing oxidoreductase is preferably selected from glucose oxidase (EC 1.1.3.4), hexose oxidase (EC 1.1.3.5), galactose oxidase (EC 1.1.3.9) and pyranose oxidase (EC 1.1.3.10). Glucose oxidase (EC 1.1.3.4) is particularly preferred according to the invention.

When using a hydrogen peroxide-producing oxidoreductase, compounds, preferably organic compounds, particularly preferably aromatic compounds that can interact with the enzymes, are also added in order to enhance the activity of the oxidoreductases in question (enhancers) or, in the event of a major difference in redox potential between the oxidizing enzymes and the soiling, to ensure electron flow (mediators).

The hydrogen peroxide-producing oxidoreductase, when used in the washing and cleaning agents according to the invention, is preferably used in a quantity such that the entire agent exhibits an oxidoreductase-related enzyme activity of 30 U/g to 20,000 U/g, in particular of 60 U/g to 15,000 U/g. The unit 1 U herein corresponds to the activity of that quantity of enzyme which converts 1 μmol of its substrate at pH 7 and 25° C. in one minute.

The substrate that is optionally used when such a hydrogen peroxide-producing oxidoreductase is used is generally indicated directly by the name of the particular oxidoreductase.

In order to achieve an improved bleaching action when washing at temperatures of 60° C. and below, and in particular in laundry pretreatment, the agents may, in addition to the metal complexes according to the invention and the additional bleach catalysts optionally present, may also contain bleach activators as an additional bleaching auxiliary. Bleach activators that may be used are compounds which, under perhydrolysis conditions, yield aliphatic peroxycarboxylic acids preferably having 1 to 10 C atoms and in particular 2 to 4 C atoms, and/or yield optionally substituted perbenzoic acid. Suitable substances are those which bear O- and/or N-acyl groups having the stated number of C atoms and/or optionally substituted benzoyl groups. Preferred compounds are repeatedly acylated alkylenediamines, in particular tetraacetylethylenediamine (TAED), acylated triazine derivatives, in particular 1,5-diacetyl-2,4-dioxohexahydro-1,3,5-triazine (DADHT), acylated glycolurils, in particular tetraacetylglycoluril (TAGU), N-acylimides, in particular N-nonanoylsuccinimide (NOSI), acylated phenolsulfonates, in particular n-nonanoyl- or isononanoyloxybenzenesulfonate (n- or iso-NOBS), carboxylic anhydrides, in particular phthalic anhydride, acylated polyhydric alcohols, in particular triacetin, ethylene glycol diacetate and 2,5-diacetoxy-2,5-dihydrofuran, n-methylmorpholinium-acetonitrile-methyl sulfate (MMA) and acetylated sorbitol and mannitol or mixtures thereof (SORMAN), acylated sugar derivatives, in particular pentaacetyl glucose (PAG), pentaacetyl fructose, tetraacetyl xylose and octaacetyl lactose and acetylated, optionally N-alkylated glucamine and gluconolactone, and/or N-acylated lactams, for example N-benzoylcaprolactam. Hydrophilically substituted acyl acetals and acyl lactams are likewise preferably used. Combinations of conventional bleach activators may also be used.

Further bleach activators that are preferably used for the purpose of the present application include compounds from the group of cationic nitriles having the general formula:

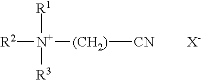

in which R¹ denotes —H, —CH₃, a C₂₋₂₄ alkyl or alkenyl residue, a substituted C₂₋₂₄ alkyl or alkenyl residue with at least one substituent from the group —Cl, —Br, —OH, —NH₂, —CN, an alkyl- or alkenylaryl residue with a C₁₋₂₄ alkyl group, or denotes a substituted alkyl- or alkenylaryl residue with a C₁₋₂₄ alkyl group and at least one further substituent on the aromatic ring, R² and R³ are mutually independently selected from —CH₂—CN, —CH₃, —CH₂—CH₃, —CH₂—CH₂—CH₃, —CH(CH₃)—CH₃, —CH₂—OH, —CH₂—CH₂—OH, —CH(OH)—CH₃, —CH₂—CH₂—CH₂—OH, —CH₂—CH(OH)—CH₃, —CH(OH)—CH₂—CH₃, —(CH₂CH₂—O)ₙH with n=1, 2, 3, 4, 5 or 6 and X is an anion.

A particularly preferred cationic nitrile is represented by the general formula:

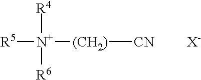

in which R⁴, R⁵ and R⁶ are mutually independently selected from —CH₃, —CH₂—CH₃, —CH₂—CH₂—CH₃, —CH(CH₃)—CH₃, in which R⁴ may additionally also be —H and X is an anion, in which preferably R⁵═R⁶═—CH₃ and in particular R⁴═R⁵═R⁶═—CH₃ applies and compounds of the formulae (CH₃)₃N⁽⁺⁾CH₂—CN X⁻, (CH₃CH₂)₃N⁽⁺⁾CH₂—CN X⁻, (CH₃CH₂CH₂)₃N⁽⁺⁾CH₂—CN X⁻, (CH₃CH(CH₃))₃N⁽⁺⁾CH₂—CN X⁻, or (HO—CH₂—CH₂)₃N⁽⁺⁾CH₂—CN X⁻ are particularly preferred, in which from the group of these substances it is in turn the cationic nitrile of the formula (CH₃)₃N⁽⁺⁾CH₂—CN X⁻, in which X₋ denotes an anion which is selected from the group chloride, bromide, iodide, hydrogensulfate, methosulfate, p-toluenesulfonate (tosylate) or xylenesulfonate which is particularly preferred.

Bleach activators are preferably used in quantities of up to 15 wt. %, preferably up to 10 wt. %, in particular 0.1 wt. % to 8 wt. %, particularly 2 to 8 wt. % and most preferably 2 to 6 wt. %, in each case relative to the total weight of the agents containing bleach activator.

Apart from at least one cyclic peptide and/or at least one metal complex of a cyclic peptide in accordance with the present invention, and the preferred bleaching agent and optional bleaching auxiliaries discussed above, a washing or cleaning agent in accordance with the present invention may also include ingredients such as additional enzymes, enzyme stabilizers, surfactants, in particular nonionic, anionic, cationic and/or amphoteric surfactants, builders, polymers, solvents, thickeners, sequestering agents, electrolytes, optical brighteners, graying inhibitors, dye transfer inhibitors, glass corrosion inhibitors, corrosion inhibitors, disintegration auxiliaries, foam inhibitors, abrasives, dyes, scents, antimicrobial active substances, UV absorbers, anticrease agents, antistatic agents, “soil release” active ingredients or “soil repellents”, propellants and/or perfume carriers, and other conventional ingredients.

With regard to builders, surfactants, further enzymes, enzyme stabilizers, polymers, solvents, thickeners, optical brighteners, graying inhibitors, anticrease agents, antistatic agents, glass corrosion inhibitors, corrosion inhibitors, “soil repellents”, dye transfer inhibitors, foam inhibitors, abrasives, disintegration agents/auxiliaries, acidifying agents, dyes, scents, antimicrobial active ingredients, UV absorbers and propellants, and the preferred quantities of these materials usable according to the invention, reference is made to published patent application WO2008/107346.

The present invention also independently provides methods for the machine cleaning of textiles or hard surfaces in which a metal complex of a cyclic peptide according to the invention is used in at least one of the steps of the method.

These include both manual and machine methods, with the machine methods preferred due to the ability to control conditions such as quantities used and period of action.

Methods for cleaning textiles are generally distinguished in that in two or more method steps various substances having cleaning action are applied onto the material to be cleaned and, after the required exposure time, are washed off. Alternatively the material to be cleaned is treated in some other manner with a washing agent or a solution of this agent. The same applies to methods for cleaning any material other than textiles, grouped together under “hard surfaces”. In exemplary embodiments of the present invention, at least one of the method steps in any conceivable washing or cleaning method may be enhanced with metal complexes described herein.

The present invention also independently provides the use of metal complexes of cyclic peptides for cleaning textiles or hard surfaces. The above-mentioned concentration ranges apply correspondingly to these uses.

Metal complexes according to the present invention may in fact be used to eliminate contaminants from textiles or hard surfaces by oxidative means. Exemplary embodiments include hand washing, manual removal of blemishes from textiles or hard surfaces, or use in connection with a machine method.

In a preferred embodiment of this use, the metal complexes according to the present invention are provided in the context of one of the above-stated formulations for washing or cleaning agents.

Exemplary embodiments of a pulverulent complete washing agent according to the present invention preferably contain components selected from the following: anionic surfactants, such as for example alkylbenzenesulfonate, alkyl sulfate, in quantities of 5-30 wt. %, preferably 8-15 wt. %, in particular 15-20 wt. %; nonionic surfactants, for example fatty alcohol polyglycol ether, alkyl polyglucoside, fatty acid glucamide, in quantities of 0.1-20 wt. %, preferably 2-15 wt. %, in particular 6-11 wt. %; builders, for example zeolite, polycarboxylate, sodium citrate, in quantities of 5-60 wt. %, preferably 10-55 wt. %, in particular 15-40 wt. %; alkalis, for example sodium carbonate, in quantities of 1-30 wt. %, preferably 2-25 wt. %, in particular 5-20 wt. %; bleaching agents, for example sodium perborate, sodium percarbonate, in quantities of 5-25 wt. %, preferably 10-20 wt. %; corrosion inhibitors, for example sodium silicate, in quantities of 1-6 wt. %, preferably 2-5 wt. %, in particular 3-4 wt. %; stabilizers, for example phosphonates, in quantities of 0-1 wt. %; foam inhibitors, for example soap, silicone oils, paraffins, in quantities of 0.1-4 wt. %, preferably 0.2-2 wt. %, in particular 1-3 wt. %; enzymes, for example proteases, amylases, cellulases, lipases, in quantities of 0.1-2 wt. %, preferably 0.2-1 wt. %, in particular 0.3-0.8 wt. %; anti-graying agents, for example carboxymethylcellulose, in quantities of 0-1 wt. %; discoloration inhibitors, for example polyvinylpyrrolidone derivatives, in quantities of 0-2 wt. %; adjusting agents, for example sodium sulfate, in quantities of 0-20 wt. %; optical brighteners, for example stilbene derivative, biphenyl derivative, in quantities of 0.1-0.3 wt. %, in particular 0.1-0.4 wt. %; scents; water; soap; bleach activators; cellulose derivatives; soil repellents; and, from 0.01 to 5 wt. %, preferably 0.02 to 4 wt. %, and in particular 0.05 to 3 wt. %, of cyclic peptides according to the invention, and/or metal complexes of these cyclic peptides, as bleach catalysts.

Exemplary embodiments of a liquid complete washing agent according to the present invention preferably contain components selected from the following: anionic surfactants, for example alkylbenzenesulfonate, alkyl sulfate, in quantities of 5-40 wt. %, preferably 8-30 wt. %, in particular 15-25 wt. %; nonionic surfactants, for example fatty alcohol polyglycol ether, alkyl polyglucoside, fatty acid glucamide, in quantities of 0.1-25 wt. %, preferably 5-20 wt. %, in particular 10-15 wt. %; builders, for example polycarboxylate, sodium citrate, in quantities of 0-15 wt. %, preferably 0.01-10 wt. %, in particular 0.1-5 wt. %; foam inhibitors, for example soap, silicone oils, paraffins, in quantities of 0.1-4 wt. %, preferably 0.2-2 wt. %, in particular 1-3 wt. %; enzymes, for example proteases, amylases, cellulases, lipases, in quantities of 0.1-2 wt. %, preferably 0.2-1 wt. %, in particular 0.3-0.8 wt. %; optical brighteners, for example stilbene derivative, biphenyl derivative, in quantities of 0.1-0.3 wt. %, in particular 0.1-0.4 wt. %; scents; stabilizers; water; soap, in quantities of 1-20 wt. %, preferably 2-15 wt. %, in particular 5-10 wt. %; alcohols/solvents, in quantities of 0-25 wt. %, preferably 1-20 wt. %, in particular 2-15 wt. %; 2 to 20 wt. %, preferably 4 to 15 wt. % and in particular 6 to 12 wt. % of bleaching agents; and, 0.01 to 5 wt. %, preferably 0.02 to 4 wt. % and in particular 0.05 to 3 wt. % of cyclic peptides according to the invention, and/or metal complexes of these cyclic peptides, as bleach catalysts.

The present invention also provides a product containing a composition according to the invention, or a washing or cleaning agent according to the invention and in particular a cleaning product for hard surfaces according to the invention, along with a spray dispenser. The product may comprise a single chamber or multichamber container, such as a two-chamber container. The spray dispenser is preferably a manually actuated spray dispenser, in particular a dispenser selected from the group encompassing aerosol spray dispensers (pressurized gas container; also known, inter alia, as a spray can), self-pressurizing spray dispensers, pump spray dispensers and trigger spray dispensers, in particular pump spray dispensers and trigger spray dispensers with a container made from transparent polyethylene or polyethylene terephthalate. Spray dispensers are described in greater detail in WO 96/04940 (Procter & Gamble) and the US patents cited therein in relation to spray dispensers, incorporated herein in its entirety by reference. Trigger spray dispensers and pump atomizers have the advantage over pressurized gas containers that no propellant is needed. Suitable attachments on the spray dispenser, e.g. nozzles etc. (“nozzle valves”) capable of passing particulates, opens up the option of adding enzymes immobilized on particles, where the agent may accordingly be dispensed as a cleaning foam.

Exemplary embodiments of an automatic dishwashing agent according to the present invention preferably comprise: from 5 to 70 wt. %, preferably 10 to 60 wt. % and in particular 20 to 50 wt. % of builder(s), with the exception of polymers with a washing and cleaning action; 2 to 28 wt. %, preferably 4 to 20 wt. % and in particular 6 to 15 wt. % of polymers with a washing and cleaning action; 0.5 to 10 wt. %, preferably 1 to 8 wt. % and in particular 2 to 6 wt. % of surfactant(s), preferably nonionic and/or amphoteric surfactant(s); 0.5 to 8 wt. %, preferably 1 to 7 wt. % and in particular 2 to 6 wt. % of enzyme(s); 2 to 20 wt. %, preferably 4 to 15 wt. % and in particular 6 to 12 wt. % of bleaching agents; and, 0.01 to 5 wt. %, preferably 0.02 to 4 wt. % and in particular 0.05 to 3 wt. % of cyclic peptides according to the invention, and/or metal complexes of these cyclic peptides, as bleach catalysts.

Automatic dishwashing agents according to the present invention may be formulated in various ways. For example, they may assume the physical form of solid, or liquid, or as a combination of solid and liquid presentations.

Suitable solid presentations include powders, granules, extrudates, or compacted products, and in particular, tablets. Liquid presentations based on water and/or organic solvents may be thickened into a gel.

Agents according to the present invention may be formulated in the form of single phase or multiphase products. Preferred automatic dishwashing agents are preferably one, two, three, or four phase products. Automatic dishwashing agents that assume the form of a prefabricated dispensing unit having two or more phases are particularly preferred.

The individual phases of multiphase agents may be of identical or different states of aggregation. Preferred automatic dishwashing agents are those which comprise at least two different solid phases, and/or at least two liquid phases, and/or at least one solid and at least one liquid phase.

Automatic dishwashing agents according to the invention are preferably preformulated as dispensing units. These dispensing units preferably comprise the quantity of substances with a washing or cleaning action required for a cleaning cycle. Preferred dispensing units have a weight of between 12 and 30 g, preferably of between 14 and 26 g and in particular of between 16 and 22 g.

The volume of the above-stated dispensing units and their three-dimensional shape are preferentially selected such that the preformulated units can be dispensed by means of the dispensing chamber of a dishwashing machine. The volume of the dispensing unit therefore preferably amounts to between 10 and 35 ml, preferably between 12 and 30 ml and in particular between 15 and 25 ml.

The automatic dishwashing agents according to the present invention, and in particular the prefabricated dispensing units, preferably comprise a water-soluble covering.

The following non-limiting examples further illustrate embodiments of the present invention:

### Example 1

### Production of Bacitracin-Metal Complex and its Bleaching Action on Tea

(a) Production of the tea solution: One liter of boiling distilled water was poured onto 1.6 g of black, unperfumed tea and left to brew for 5 minutes. The insoluble components were then filtered out through a fluted filter, and after cooling to room temperature, the pH value was adjusted to 10.0 with sodium hydroxide solution. The solution thus prepared may be used for a period of 1-2 days.

(b) Production of the bacitracin-metal complexes: 0.025 mmol (relative to the metal atom) of the metal salt to be investigated was stirred with 0.050 mmol of bacitracin for up to 24 hours at room temperature in an open vessel in 25 ml of deionized water. The pH was adjusted to 10 and the mixture left to stand for 48 hours at room temperature, after which the insoluble components were removed by sterile filtration through a syringe filter. A metal-free solution of bacitracin was prepared in exactly the same way, but not combined with metal salt.

(c) Measurement of bleaching performance: A hydrogen peroxide solution was freshly prepared from 1050 μL of 30% Perhydrol in 100 ml of a conventional commercial washing agent containing borate buffer. The metal complexes were diluted with the washing agent containing 50 mM borate buffer, pH 10.0, to 5.88 mg of metal per liter.

TABLE 1
Pipetting Scheme.

| | Zeroing | Uncataylzed | Complex- |
|---|---|---|---|
| | Solution | Bleach | catalyzed |
| Tea solution | 400 μL | 400 μL | 400 μL |
| Washing agent with borate buffer | 100 μL | 100 μL | 100 μL |
| Deionized water | 500 μL | 400 μL | 300 μL |
| H₂O₂ Solution | 0 μL | 100 μL | 100 μL |
| Bactiracin-metal complex solution | 0 μL | 0 μL | 100 μL |

The hydrogen peroxide solution was added last, before the batches were sealed and incubated for 1-hour at 40° C. Absorbance was then measured at 400 nm relative to water in the spectrophotometer. The decrease in absorbance AE minus that of the uncatalyzed bleach is a measure of bleaching performance and indicates how strongly the tested complex was able to promote hydrogen peroxide bleaching.

Bleaching performance was evaluated by setting the bleaching performance of the solution with hydrogen peroxide without catalyst to 100%, such that values over 100 were indicative of better bleaching performance, while values below 100 were indicative of poorer bleaching performance, compared to the performance of the washing agent with hydrogen peroxide but without catalyst.

TABLE 2
Bleaching Performance of Various Bacitracin-Metal Complexes.

| Central atom | Ce | Eu | Ti | VOSO₄ | Na₃VO₄ | Cr | Mo | W | Mn(II) | Mn(III) |
|---|---|---|---|---|---|---|---|---|---|---|
| Bleaching performance | 136 | 110 | 115 | 141 | 133 | 136 | 140 | 140 | 141 | 143 |

| Central atom | Fe(II) | Fe(III) | Co | Ni | Cu | Zn | Al | Ga | Bi |
|---|---|---|---|---|---|---|---|---|---|
| Bleaching performance | 135 | 99 | 342 | 117 | 130 | 148 | 115 | 120 | 133 |

As shown in TABLE 2, it is possible to bring about a distinct improvement in bleaching performance with many different bacitracin-metal complexes. The sharp increase in bleaching performance when using complexes of bacitracin with cerium, vanadium, chromium, molybdenum, tungsten, manganese, iron (II), cobalt, zinc and bismuth ions is particularly striking.

We claim:

1. A washing or cleaning agent comprising an oligopeptide, said oligopeptide further comprising (a) at least one thiazole residue, said thiazole residue optionally partially or completely hydrogenated; and (b) at least one imidazole residue, said imidazole residue optionally partially or completely hydrogenated.

2. The washing or cleaning agent of claim 1 wherein the oligopeptide comprises 5 to 30 amino acids.

3. The washing or cleaning agent of claim 1 wherein the oligopeptide comprises at least one intrachain covalent linkage.

4. The washing or cleaning agent of claim 3 wherein the oligopeptide comprises a cyclic hexa-, hepta- or octapeptide.

5. The washing or cleaning agent of claim 1 wherein the oligopeptide comprises at least one amino acid comprising a carboxyl group and/or carboxamide group in its side chain.

6. The washing or cleaning agent of claim 1 wherein the oligopeptide comprises 10 to 20 amino acids that include at least one lysine residue, and wherein (a) the seven C-terminal amino acids of said oligopeptide form a cyclic heptapeptide by means of an epsilon-peptide bond between the C-terminal amino acid and the side chain of a lysine residue; and (b) wherein two successive amino acids in the N-terminal region of said oligopeptide form a thiazoline residue outside of said cyclic heptapeptide.

7. The washing or cleaning agent of claim 1 wherein the oligopeptide is synthesized naturally by nonribosomal peptide synthetases.

8. The washing or cleaning agent of claim 1 wherein the oligopeptide is bacitracin or a derivative of bacitracin, said derivative obtainable from bacitracin by substitution of up to five amino acids, or by addition of up to five amino acids in the N-terminal region of bacitracin.

9. The washing or cleaning agent of claim 1 further comprising a metal ion that forms a metal complex with said oligopeptide.

10. The washing or cleaning agent of claim 9 wherein said metal ion is any oxidation state of a metal selected from the group consisting of Ag, Al, Au, B, Bi, Ce, Co, Cr, Cu, Eu, Fe, Ga, Hg, Mn, Mo, Ni, Pb, Pt, Ru, Ti, U, V, W, Zn, and Zr, and mixtures thereof.

11. The washing or cleaning agent of claim 9 wherein the metal ion of the metal complexes of the oligopeptide is a transition metal ion.

12. The washing or cleaning agent of claim 11 wherein the transition metal ion is selected from the group consisting of Fe(III), Co(II), Co(III), Mn(II), Mn(III), Mn(IV), Ce(III), Ce(IV), Zn(II), and Bi(III), and mixtures thereof.

13. The washing or cleaning agent of claim 1 further comprising at least one surfactant.

14. The washing or cleaning agent of claim 1 further comprising at least one bleaching agent.

15. The washing or cleaning agent of claim 1 wherein said agent is entirely devoid of a bleaching agent.

16. The washing or cleaning agent of claim 1 further comprising at least one hydrogen peroxide-producing oxidoreductase.

17. The washing or cleaning agent of claim 1 wherein said agent is a fiber care and/or color care agent.

18. A method of cleaning soiled hard surfaces comprising the step of applying the washing or cleaning agent of claim 1 to the soiled surface.

19. A method of cleaning soiled hard surfaces comprising the step of applying the washing or cleaning agent of claim 9 to the soiled surface.

20. A method of cleaning soiled hard surfaces comprising the step of applying the washing or cleaning agent of claim 14 to the soiled surface.

21. A method of cleaning textile fabrics comprising the step of applying the washing or cleaning agent of claim 1 to said textile fabric.

22. A method of cleaning textile fabrics comprising the step of applying the washing or cleaning agent of claim 9 to said textile fabric.

23. A method of cleaning textile fabrics comprising the step of applying the washing or cleaning agent of claim 14 to said textile fabric.

24. The method of claim 18 wherein said soil is selected from the group consisting of tea, coffee, blood, soot, egg, milk, butter, ink, makeup, lipstick, chocolate, chocolate dessert, olive oil, black currant, blueberry, apple juice, red wine, beetroot, curry, and peanut oil, and mixtures thereof.

25. A method for bleaching wood pulp or raw cotton comprising the step of treating said wood pulp or raw cotton with the washing or cleaning agent of claim 1.

26. A method for bleaching wood pulp or raw cotton comprising the step of treating said wood pulp or raw cotton with the washing or cleaning agent of claim 9.

27. A method for bleaching wood pulp or raw cotton comprising the step of treating said wood pulp or raw cotton with the washing or cleaning agent of claim 14.

28. The method of claim 19, wherein said oligopeptide-metal complex is produced in situ.

29. The method of claim 22, wherein said oligopeptide-metal complex is produced in situ.

30. The method of claim 26, wherein said oligopeptide-metal complex is produced in situ.

31. The method according to claim 28 wherein said washing or cleaning agent comprises separate oligopeptide-containing and metal ion-containing phases.

32. The method according to claim 29 wherein said washing or cleaning agent comprises separate oligopeptide-containing and metal ion-containing phases.

33. The method according to claim 30 wherein said washing or cleaning agent comprises separate oligopeptide-containing and metal ion-containing phases.

34. A method for killing microbes comprising the step of contacting said microbes with the washing or cleaning agent of claim 1.

35. An oligopeptide-metal complex comprising:
(a) an oligopeptide further comprising:
(i) at least one thiazole residue, said thiazole residue optionally partially or completely hydrogenated;
(ii) at least one imidazole residue, said imidazole residue optionally partially or completely hydrogenated; and
(b) a metal selected from the group consisting of Ag, Al, Au, B, Bi, Ce, Co, Cr, Cu, Eu, Fe, Ga, Hg, Mn, Mo, Ni, Pb, Pt, Ru, Ti, U, V, W, Zn, and Zr, and mixtures thereof; and
wherein said metal is present in any oxidation state, and wherein said oligopeptide-metal complex is not Zn-bacitracin.

36. The oligopeptide-metal complex of claim 35 wherein said metal is selected from the group consisting of Co(II), Co(III), Mn(II), Mn(III), Ce(III), Ce(IV), and Bi(III), and mixtures thereof.

37. The oligopeptide-metal complex of claim 35 wherein said oligopeptide is bacitracin, and said metal is selected from the group consisting of Ag, Al, Au, B, Bi, Ce, Co, Cr, Cu, Eu, Fe, Ga, Hg, Mn, Mo, Ni, Pb, Pt, Ru, Ti, U, V, W, and Zr, and mixtures thereof, and where said metal is present in any oxidation state.

38. The oligopeptide-metal complex of claim 35 wherein said oligopeptide is a bacitracin derivative, said derivative obtainable from bacitracin by substitution of up to five amino acids, or by addition of up to five amino acids in the N-terminal region of bacitracin.
